(19) United States
(12) Patent Application Publication
(10) Pub. No.: US 2024/0197193 A1
(43) Pub. Date: Jun. 20, 2024

(54) LIVESTOCK HEART RATE MONITORING

(71) Applicant: The Main Branch, Inc., WOODINVILLE, WA (US)

(72) Inventors: Octavian Alexandru BLAGA, CHATTANOOGA, TN (US); David Benjamin SCOTT, WOODINVILLE, WA (US)

(21) Appl. No.: 18/537,889

(22) Filed: Dec. 13, 2023

Related U.S. Application Data
(60) Provisional application No. 63/387,488, filed on Dec. 14, 2022.
(60) Provisional application No. 63/387,491, filed on Dec. 14, 2022.
(60) Provisional application No. 63/387,490, filed on Dec. 14, 2022.

(51) Int. Cl. A61B 5/024; A01K 11/00; A01K 29/00; A61B 5/00; G06T 5/70; G06T 7/00

(52) U.S. Cl. CPC A61B 5/02416; A01K 11/006; A01K 29/005; A61B 5/742; G06T 5/70; G06T 7/0012; A61B 2503/40; G06T 2207/10016; G06T 2207/20221; G06T 2207/30004

(57) ABSTRACT

According to embodiments, a computer method for monitoring livestock heart rate processes received (optionally hyperspectral) video images to measure a feature on the livestock subject to periodic flushing correlated to heartbeat period. The method applies image processing including identifying a location of the feature, applying pixel amplification to increase periodic light scattering or emission variations, and calculating a heart rate corresponding to a detected period of the flushing.

### CROSS REFERENCE TO RELATED APPLICATIONS

The present application claims benefit of U.S. Provisional Patent Application No. 63/387,488, entitled “LIVESTOCK HEART RATE MONITORING”, filed Dec. 14, 2022 (Docket No.: 3087-004-02); U.S. Provisional Patent Application No. 63/387,491, entitled “METHOD AND SYSTEM FOR DETECTING LIVESTOCK RESPIRATORY COMPROMISE”, filed Dec. 14, 2022 (Docket No.: 3087-003-02); and U.S. Provisional Patent Application 63/387,490, entitled “COMPUTER METHOD AND APPARATUS FOR TAGLESS TRACKING OF LIVESTOCK”, filed Dec. 14, 2022 (Docket No. 3087-005-02), co-pending herewith.

The foregoing applications, to the extent not inconsistent with the disclosure herein, are incorporated by reference.

### SUMMARY

According to an embodiment, a computer method for measuring heart rate in livestock includes receiving a video stream including one or more livestock in a pen; determining, from the video stream, a digital identity of a livestock individual; finding, in frames of the video stream, a feature of the livestock individual that exhibits heart rate-correlated periodic flushing; performing pixel amplification at the feature to amplify the periodic flushing; and calculating a heart rate of the livestock individual from a sequence of the amplified periodic flushing. The computer method may further include outputting information about the heart rate of the livestock individual on a physical device.

### BRIEF DESCRIPTION OF THE DRAWINGS

FIG. 1 is a diagram of a system for monitoring livestock health, according to an embodiment.

FIG. 2 is a block diagram of a computer system for tagless tracking and biometric identification of livestock, according to an embodiment.

FIG. 3 is a flowchart showing a computer method for livestock biometric identification and tracking, according to an embodiment.

FIG. 4 is a diagram of information including livestock milling about with respective electronic identities output to an electronic display, according to an embodiment.

FIG. 5 is a diagram of an individual livestock with biometric markers for determining a biometric identity, according to an embodiment.

FIG. 6 is a diagram showing an electronic display where an electronic identity is adjusted to correspond to a biometric identity, according to an embodiment.

FIG. 7 is a flow chart showing a computer method for measuring heart rate in livestock, according to an embodiment.

FIG. 8 is a diagram showing a livestock individual with a feature that exhibits periodic flushing with heartbeat, according to an embodiment.

FIG. 9 is a diagram showing a sequence of portions of video frames exhibiting pixel-amplified periodic flushing at the feature of the livestock, according to an embodiment

### DETAILED DESCRIPTION

In the following detailed description, reference is made to the accompanying drawings, which form a part hereof. In the drawings, similar symbols typically identify similar components, unless context dictates otherwise.

Other embodiments may be used and/or other changes may be made without departing from the spirit or scope of the disclosure.

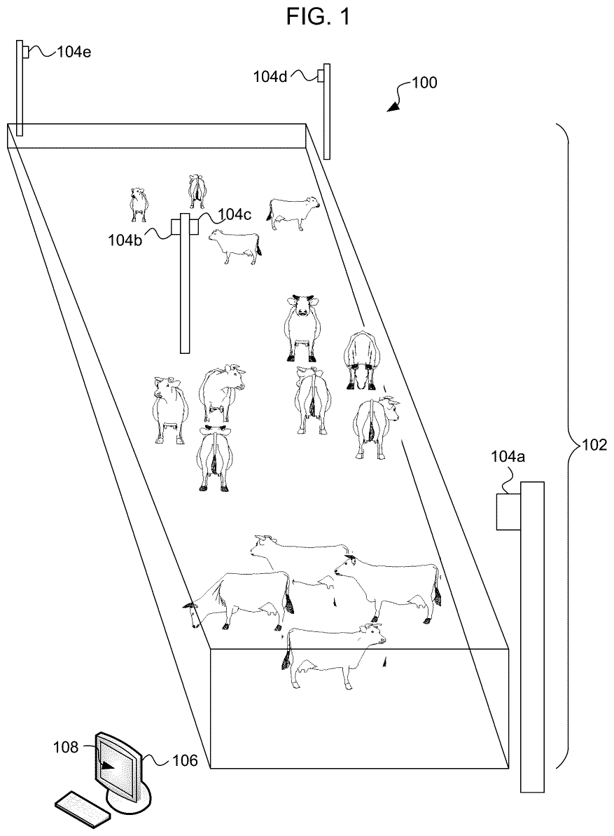

FIG. 1 is a diagram of a system 100 for monitoring livestock health, according to an embodiment. A plurality of livestock 102 may be constrained by a peripheral fence, for example in a feedlot. The computer methods described herein may be performed on such a peripherally constrained plurality of livestock, or may be performed on livestock with no nearby constraint.

A plurality of sensors 104, here shown as cameras 104a, 104b, 104c, 104d, 104e, are disposed to obtain digital video or sequences of still frames including the livestock 102. The digital video or sequences of still frames are transmitted to a computer 106. The computer 106 may process the digital video or sequences of still frames as described below. The computer 106 may display the videos or sequences of still frames on an electronic display 108 for viewing by a user. Additionally or alternatively the computer 106 may display other indicia, optionally overlaying the fields of view, derived from processing described below.

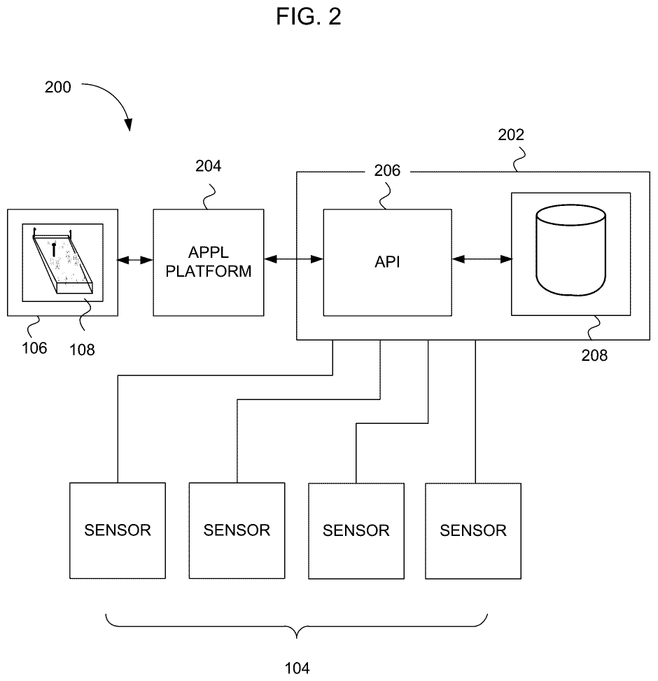

FIG. 2 is a block diagram of a computer system for tag-less tracking and biometric identification of livestock, according to an embodiment. The cameras 104a, 104b, 104c, 104d, 104e are indicated as sensors 104. The sensors 104 may optionally include other sensing modalities in addition to focal plane imaging. Optionally, the sensors 104 are configure to provide hyper-spectral imaging. The sensors are operatively coupled to a computer 202. As shown in FIG. 2, the computer 106 shown in FIG. 1 may be configured as a client or peer device.

Optionally all processing described herein may be performed in a single computer 106. The computer 106 may include a thin client, a portable or non-portable computer, a personal electronic device such as a smart phone, or other platform capable of receiving data and driving an electronic display 108.

The computer 202 may be a server computer, and/or may include a server farm, a set of pipelined servers, relay servers, etc. as is known in the art of computer networking. The server 202 may receive data from the sensors 104 and process the data as describe herein. The server may provide an application programming interface (API) portion 206 operatively coupled to an application platform 204. The application platform 204 may be included in the server 202, may be included in the local computer 106, or may be otherwise operatively coupled therebetween.

The server 202 and/or computer 106 includes a non-transitory computer readable memory 208 such as a rotating disk or solid state memory. The non-transitory computer readable memory may support a database, look-up table, or other software structure to enable storage and retrieval of information described below. Typically, the computer 202 includes a microprocessor, memory, and other components appropriate for performing image processing on the data received from the sensors 104.

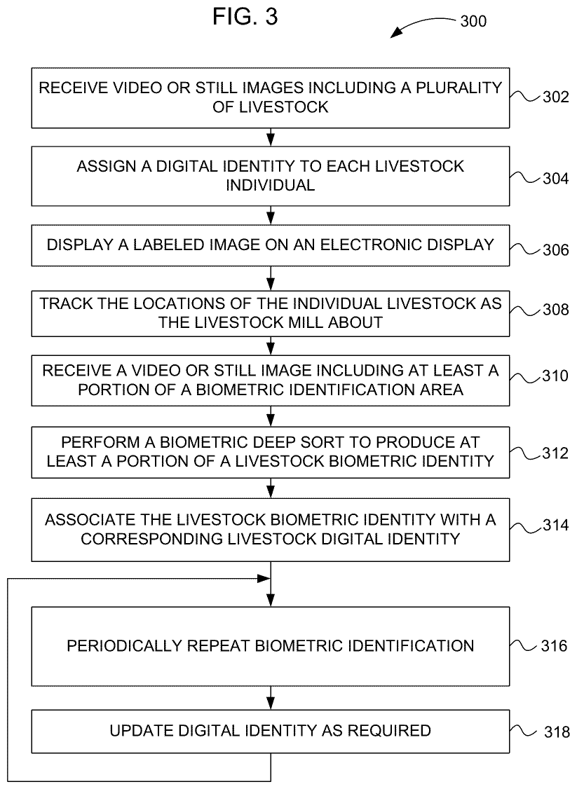

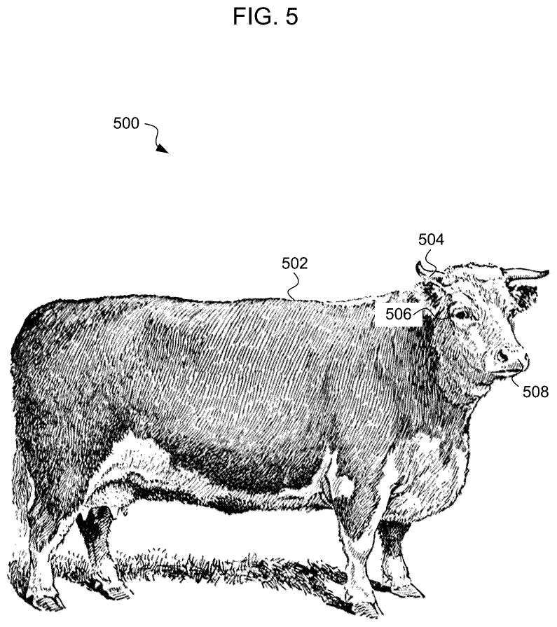

FIG. 3 is a flow chart showing a computer method 300 for livestock tracking and biometric identification, according to an embodiment which includes, at step 302, receiving, into a computer 106, 202, a first digital video or sequence of digital photographic frames from one or more digital image capture devices 104a, 104b, 104c, 104d, 104e. The digital image capture devices 104a, 104b, 104c, 104d, 104e may be referred to, collectively or individually, as 104 herein. The first digital video or sequence of digital photographic frames may include a plurality of livestock locations in a pen 102. Proceeding to step 304, a digital identity is assigned to each livestock individual in the pen. In step 308, locations of the livestock individuals are tracked as the livestock mill about. Referring to FIGS. 3 and 5, the method 300 may further include, at step 310, receiving a second digital video or digital photographic frame 500 including at least a portion of a biometric identification area 504, 506, 508 of an individual livestock body 502. A biometric deep sort is performed in step 312 to produce at least a portion of an individual livestock biometric identity. Proceeding to step 314, the individual livestock biometric identity is associated with a corresponding livestock digital identity. Step 314 may, for example, include associating the biometric and digital identities in a look up table, database, or other logical construct saved onto a non-transitory computer readable medium.

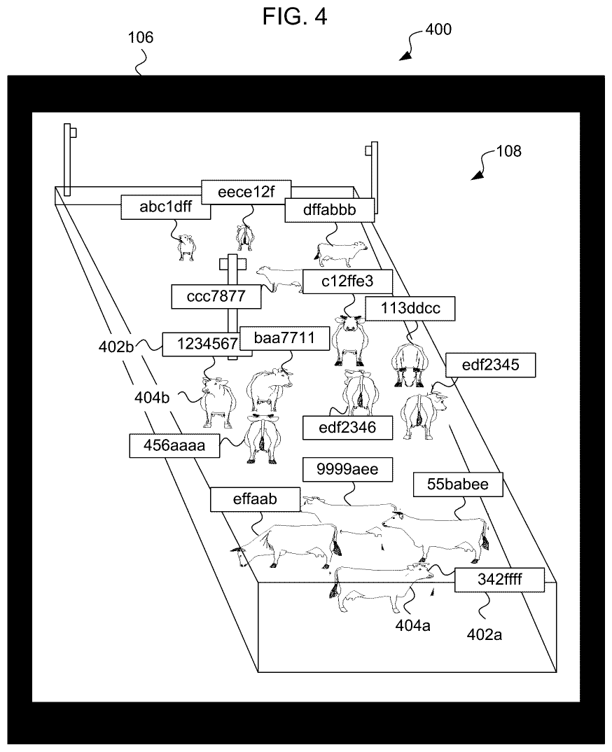

Referring to FIGS. 3 and 4, the method 300 may further include, in step 306, displaying a labeled image 400 on an electronic display 108, the labeled image including the current digital identities 402 corresponding each livestock individual.

According to an embodiment, tracking locations of the livestock individuals as the livestock mill about, in step 308, is performed using a Kalman filter. According to embodiments, tracking the locations of the livestock individuals may employ at least some parts of simultaneous localization and mapping (SLAM) which uses the Kalman filter. SLAM is usually used in robotics so the robot knows where in the environment it is located based on a few measurements.

In standard SLAM, the robot sends some lasers or pings in the environment to figure out where it is, based on a few reference points. In the present embodiment, if one uses multiple poles, each supporting a digital camera or video device, the pole locations serve as reference points from which the computer method, and specifically step 308, obtains measurements of the location and velocity of each detected class. In this case, the detected class is “livestock”. With measurements from one or many poles, step 308 includes calculating a probability that a particular detected livestock individual is the same detected livestock individual from a previous few frames. The more poles there are, the more accurate the measurements will be for both tracking and identification.

Referring to FIG. 5, receiving, in step 310, the second digital video or digital photographic frame including at least a portion of a biometric identification area of an individual livestock body may include receiving a plurality of frames. According to an embodiment, the computer method proceeds to step 312, which includes performing the biometric deep sort. Step 312 may include operating a Harris filter to locate livestock biometric markers. The Harris filter identifies corners in the frame, the corners being associated with the biometric markers. Performing the biometric deep sort in step 312 may include identifying, on a grid, relative locations of individual livestock biometric markers and storing the grid locations in an individual livestock biometric identity record in a livestock population model.

The individual livestock biometric markers may include at least two of a corner of an eye, a corner formed by a horn, a corner formed by an ear, a snout corner, a hide color corner, a hoof corner, and/or a tail corner. Identifying individual biometric markers may include assigning classes of livestock body surface features and performing a semantic segmentation to classify each pixel as belonging to a livestock body surface feature.

Assigning classes of livestock body surface features may include assigning livestock eye, livestock horn, livestock ear, livestock snout, livestock hide color patterns, livestock hoof, and/or livestock ear classes. Assigning classes of livestock body surface features may include assigning livestock eye corners and assigning livestock snout corners. Assigning classes of livestock body surface features may include assigning contrasting locations of skin and/or fur coloration.

To perform identification, the computer method includes receiving a high resolution snapshot at a given time at which heads, eyes, and snouts are detected. Step 312 may include computing all face geometries.

In step 314, the computer method 300 may include performing a probability computation against the database to get the most likely candidate electronic IDs corresponding to the biometric IDs. If a good candidate is obtained, the ID of that candidate may be assigned to the data recorded for the aforementioned detection. If multiple candidates are obtained, the multiple candidates, as well as secondary candidates, may be set to be reviewed during subsequent algorithm improvement iterations.

The first digital video or sequence of digital photographic frames received in step 302 may include a wider angle view including a plurality of livestock in the frame, compared to the second digital video or sequence of digital photographic frames includes a narrower angle view that includes less than all of the plurality of livestock in the frame. The narrower angle view may primarily include at least a portion of an individual livestock. In an embodiment, the narrower angle view consists essentially of the biometric identification area (e.g., see FIG. 5, 504, 506, 508) of the individual livestock's body 502.

The method 300 may further include receiving a third digital video or sequence of digital photographic frames including plurality of livestock locations in the pen as the livestock mill about, the plurality of livestock having corresponding digital identities and at least a portion of the plurality of livestock having been assigned a biometric identity. The individual livestock may be tracked (see step 308) as the livestock mill about, the individual livestock nominally being assigned livestock digital identities.

The method 300 may further include receiving the second digital video or digital photographic frame corresponding to one of the individual livestock and including at least a portion of the biometric identification area of the individual livestock body, performing, in step 316, a second biometric identification of the individual livestock; and, in step 318, verifying that the tracked individual livestock is the individual livestock associated with the current livestock digital identity.

According to an embodiment, the method 300 may include tracking the individual livestock as the livestock mill about in step 308, the individual livestock nominally being assigned livestock digital identities, receiving the second digital video or digital photographic frame corresponding to one of the individual livestock and including at least a portion of the biometric identification area of the individual livestock body (see step 310), and performing biometric identification of the individual livestock.

The method 300 may include determining the individual livestock does not match a biometric identity of a known individual livestock. If an individual livestock has not been biometrically identified, the method 300 may include performing the biometric deep sort (see step 312) to produce at least a portion of an individual livestock biometric identity, and (referring to step 314), associating the individual livestock biometric identity with the corresponding livestock digital identity. This may be used to gradually match individual livestock digital identities to individual livestock biometric identities after beginning tracking the livestock as the livestock mill about.

Improvement of the biometric identity may be obtained by determining that the biometric identity includes biometric markers not previously included in the biometric identity of the individual livestock (not shown) and augmenting the biometric identity with the additional biometric markers.

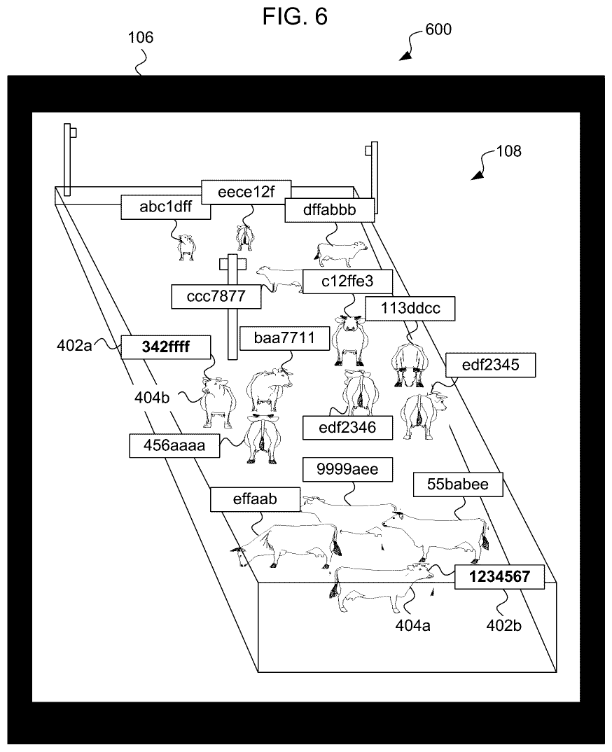

As may be appreciated with reference to FIG. 6, the individual livestock may occasionally be partially or completely obscured as the livestock mill about. This may cause the image processing software to, on occasion, lose certainty as to correct digital identities, thereby causing a reduction of correspondence between current digital identities and biometric identities of the livestock. To overcome this, the computer method may include periodically repeating biometric identification and, as appropriate, updating the digital identity.

The method 300 may include determining that the tracked individual livestock 404a is an individual livestock associated with a different individual livestock digital identity 402b than an incorrect livestock digital identity 402a currently assigned to the first individual livestock 404a. The method 300 may then include, in step 318, assigning the correct individual livestock digital identity to the individual livestock.

For example, comparing FIG. 4 to FIG. 6, see that individual livestock 404a, was incorrectly associated with digital identity 402a “342ffff”. Upon the biometric deep sort, the individual livestock 404a, was found to have been assigned the digital identity associated with a different livestock 404b. Step 318 may include assigning the correct livestock digital identity 402b “1234567” previously associated with a different individual livestock 404b than the individual livestock 404a. Step 318 may include assigning the incorrect livestock digital identity 402a “342ffff” previously associated with the first individual livestock 404a to a second individual livestock 404b.

The livestock may be cows and/or steers, for example.

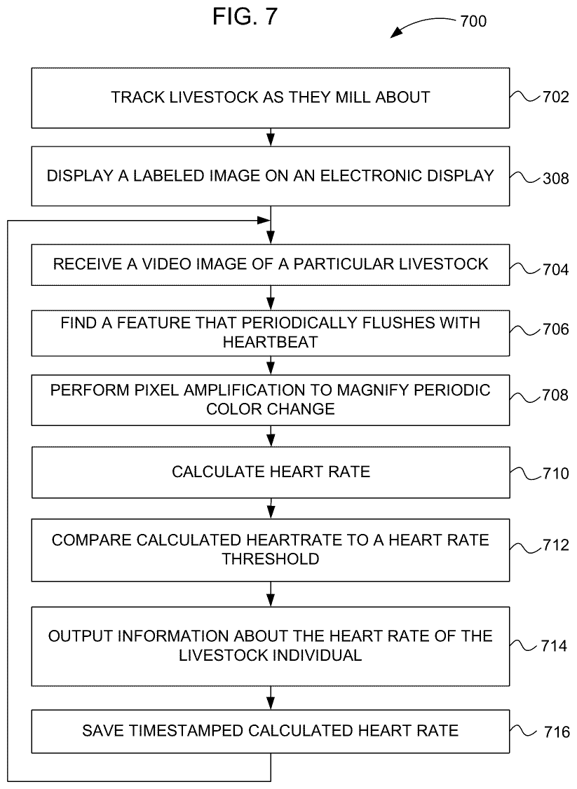

FIG. 7 is a flowchart showing a computer method 700 for measuring heart rate in livestock, according to an embodiment. Step 702 includes receiving a video stream including one or more livestock in a pen (e.g. see FIG. 1). In step 704, a digital identity of a livestock individual is determined from the video stream (e.g. see FIG. 4).

Step 706 includes using image processing to find, in frames of the video stream, a feature of the livestock individual that exhibits heart rate-correlated periodic flushing. In step 708, pixel amplification at the feature to amplify the periodic flushing is performed. Step 710 includes calculating a heart rate of the livestock individual from a sequence of the amplified periodic flushing. In step 714, information about the heart rate of the livestock individual is output on a physical device.

Receiving a video stream including one or more livestock in a pen in step 702 may include tracking livestock as they mill about. Referring to FIGS. 3 and 4, in step 306 of the method 300, a labeled image 108 of the livestock in the pen may be displayed.

Referring again to FIG. 7, receiving a video stream including the one or more livestock in the pen in step 702 may include receiving a video stream from a hyperspectral digital image capture device. Outputting information about the heart rate of the livestock individual in step 714 may include physically outputting information corresponding to the heart rate of the livestock individual. For example, outputting information about the heart rate of the livestock individual in step 714 may include displaying, on an electronic display, a notice that the individual livestock has a heart rate that meets a threshold heart rate indicative of physical distress. In other embodiments, physically outputting information corresponding to the heart rate includes sounding an audible alarm, driving a haptic device, transmitting application or browser data to a personal electronic device such that the personal electronic device displays indicia on it electronic display, outputs an audible signal, or outputs a haptic signal.

Outputting information about the heart rate of the livestock individual on an electronic display in step 714 may include displaying an image, on an electronic display, of the individual livestock exhibiting the heart rate that meets a threshold heart rate indicative of physical distress. Outputting information about the heart rate of the livestock individual in step 714 may include displaying, on an electronic display, an image of the one or more livestock as they mill about with the image of the individual livestock 404 exhibiting a heart rate highlighted. For example, see the illustrative display 400, 600 in FIGS. 4 and 6, in which two individual livestock 404a, 404b, “1234567” and “342ffff”, are labeled with highlighted digital identities 402a, 402b on an electronic display 108. Accordingly, an individual livestock exhibiting physical distress, the cause of which may include communicable disease, may be quickly identified and removed from the pen. Outputting information about the heart rate of the livestock individual on an electronic display in step 714 includes outputting a message on a personal electronic device.

In step 716, the calculated heart rate may be stored in a computer readable non-transitory memory as part of a set of related data including previously calculated heart rates of the livestock individual.

Comparing the calculated heart rate of the livestock individual to a heart rate threshold in step 712 may include comparing the calculated heart rate of the livestock individual to one or more previously calculated heart rates of the livestock individual, and determining if the calculated heart rate heart rate is different than the previously calculated heart rates.

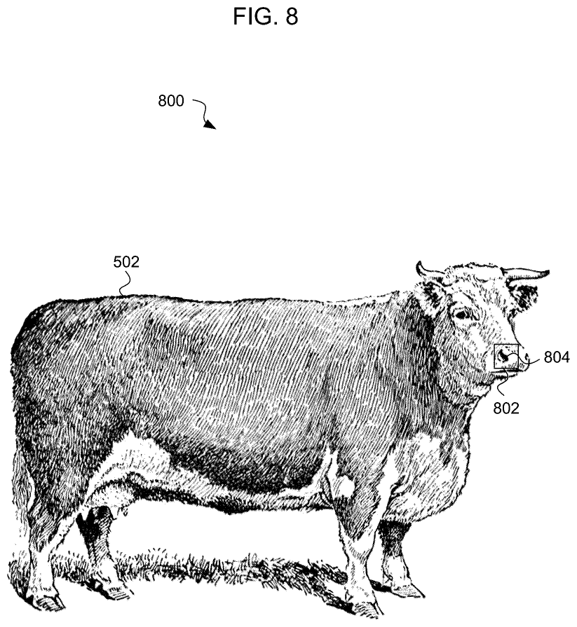

FIG. 8 is a diagram showing a feature 804 of a livestock individual 502 that exhibits periodic flushing with heartbeat. Finding the feature 804 that periodically flushes with heartbeat, in step 706, may include producing a zoomed image 802 comprising a portion of the livestock individual 502. Finding the feature that periodically flushes with heartbeat in step 706 may include, for example, finding a snout 804 of the livestock individual 502.

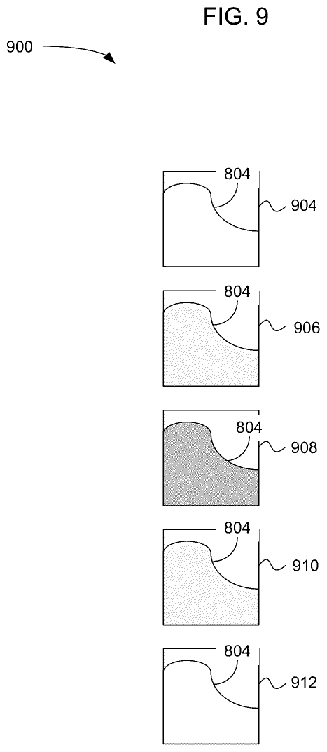

FIG. 9 is a diagram 900 showing a sequence of video frames 904, 906, 908, 910 exhibiting pixel-amplified periodic flushing at the feature 804 of the livestock, according to an embodiment. Performing pixel amplification at the feature 804 to amplify the periodic flushing in step 708 may be performed using a Laplacian Pyramid. Using the Laplacian Pyramid may include creating negatives of each image at various resolutions and performing image addition with the original image on a frame-to-frame basis.

Performing pixel amplification at the feature 804 to magnify periodic flushing in step 708 may include removing high frequency noise from an image addition frame sequence using a maximally flat magnitude filter within a passband corresponding to livestock heart rate range. In an embodiment, the maximally flat magnitude filter within the passband comprises a Butterworth filter. In other embodiments, the maximally flat magnitude filter within the passband may include a Chebyshev filter or an elliptical filter. Performing pixel amplification in step 708 may include performing a Laplacian Pyramid and applying a Butterworth filter to form a video stream with greater dynamic range and/or higher sensitivity to flushing than a video stream where pixel amplification was not performed.

In step 710, calculating the heart rate may include performing image analysis on one or more pixel-amplified video frame sequences. Determining heart rate in step 710 may include obtaining at least three video clips of pixel-amplified intervals and applying a filter to ensure the calculated heart rate is representative of an actual real time heart rate of the livestock individual. Applying the filter to ensure the calculated heart rate is representative of an actual heart rate of the livestock individual may include applying a voting algorithm or performing heart rate averaging.

While various aspects and embodiments have been disclosed herein, other aspects and embodiments are contemplated. The various aspects and embodiments disclosed herein are for purposes of illustration and are not intended to be limiting, with the true scope and spirit being indicated by the following claims.

What is claimed is:

1. A computer method for measuring heart rate in livestock, comprising:
receiving a video stream including one or more livestock;
determining, from the video stream, a digital identity of a livestock individual;
finding, in frames of the video stream, a feature of the livestock individual that exhibits heart rate-correlated periodic flushing;
performing pixel amplification at the feature to amplify the periodic flushing;
calculating a heart rate of the livestock individual from a sequence of the amplified periodic flushing; and
outputting information about the heart rate of the livestock individual on a physical device.

2. The computer method for measuring heart rate in livestock of claim 1,
wherein receiving a video stream including one or more livestock in a pen includes tracking livestock as they mill about.

3. The computer method for measuring heart rate in livestock of claim 1, further comprising:
displaying a labeled image of the livestock on an electronic display.

4. The computer method for measuring heart rate in livestock of claim 1, wherein receiving a video stream including the one or more livestock in the pen includes receiving a video stream from a hyperspectral digital image capture device.

5. The computer method for measuring heart rate in livestock of claim 1, wherein outputting information about the heart rate of the livestock individual includes physically outputting information corresponding to the heart rate of the livestock individual.

6. The computer method for measuring heart rate in livestock of claim 1, wherein outputting information about the heart rate of the livestock individual includes displaying, on an electronic display, a notice that the individual livestock has a heart rate that meets a threshold heart rate indicative of physical distress.

7. The computer method for measuring heart rate in livestock of claim 1, wherein outputting information about the heart rate of the livestock individual on an electronic display includes displaying an image, on an electronic display, of the individual livestock exhibiting the heart rate that meets a threshold heart rate indicative of physical distress.

8. The computer method for measuring heart rate in livestock of claim 1, wherein outputting information about the heart rate of the livestock individual includes displaying, on an electronic display, an image of the one or more livestock as they mill about with the imaged of the individual livestock exhibiting a heart rate highlighted.

9. The computer method for measuring heart rate in livestock of claim 1, wherein outputting information about the heart rate of the livestock individual on an electronic display includes outputting a message on a personal electronic device.

10. The computer method for measuring heart rate in livestock of claim 1, further comprising:
storing the calculated heart rate in a computer readable non-transitory memory including previously calculated heart rates of the livestock individual.

11. The computer method for measuring heart rate in livestock of claim 1, wherein comparing the calculated heart rate of the livestock individual to a heart rate threshold includes:
comparing the calculated heart rate of the livestock individual to one or more previously calculated heart rates of the livestock individual; and
determining if the calculated heart rate is different than the previously calculated heart rates; and
wherein physically outputting the information corresponding to the heart rate of the first livestock individual.

12. The computer method for measuring heart rate in livestock of claim 1, wherein finding the feature that periodically flushes with heartbeat includes finding a snout of the livestock individual.

13. The computer method for measuring heart rate in livestock of claim 1, wherein performing pixel amplification at the feature to amplify the periodic flushing is performed using a Laplacian Pyramid.

14. The computer method for measuring heart rate in livestock of claim 13, wherein using the Laplacian Pyramid includes:
creating negatives of each image at various resolutions; and
performing image addition with the original image on a frame-to-frame basis.

15. The computer method for measuring heart rate in livestock of claim 1, wherein performing pixel amplification at the feature to magnify periodic flushing further comprises:
removing high frequency noise from an image addition frame sequence using a maximally flat magnitude filter within a passband corresponding to a livestock heart rate range.

16. The computer method for measuring heart rate in livestock of claim 1, wherein performing pixel amplification includes performing a Laplacian Pyramid and applying a Butterworth filter to form a video stream having a higher sensitivity to flushing than not performing pixel amplification.

17. The computer method for measuring heart rate in livestock of claim 1, wherein calculating the heart rate includes performing image analysis on one or more pixel-amplified video frame sequences.

18. The computer method for measuring heart rate in livestock of claim 1, wherein determining heart rate includes:
obtaining at least three video clips of pixel-amplified intervals; and
applying a filter to ensure the calculated heart rate is representative of an actual heart rate of the livestock individual.

19. The computer method for measuring heart rate in livestock of claim 18, wherein applying the filter to ensure the calculated heart rate is representative of an actual heart rate of the livestock individual includes applying a voting algorithm or performing heart rate averaging.
